SEQUENCE LISTING (30 sequences)

SEQ ID NO: 1 - DNA, 34 bp, artificial sequence
  Oligonucleotides corresponding to WT ABCA1 DR4 element
actgggctttgaccgatagtaacctctgcgctcg

SEQ ID NO: 2 - DNA, 34 bp, artificial sequence
  Oligonucleotides corresponding to mutant ABCA1 DR4 element
actgggctttgtgtgatagtactatctgcgctcg

SEQ ID NO: 3 - DNA, 27 bp, artificial sequence
  Oligonucleotides used in EMSA analysis of DR4 element a
actgcagtgaccgccagtaaccccagc

SEQ ID NO: 4 - DNA, 27 bp, artificial sequence
  Oligonucleotides used in EMS analysis of DR4 element b
actgggacgcccgctagtaaccccggc

SEQ ID NO: 5 - DNA, 28 bp, mouse
ggtttggagatggttatacaatagttgt

SEQ ID NO: 6 - DNA, 17 bp, mouse
cccggaaacgcaagtcc

SEQ ID NO: 7 - DNA, 25 bp, mouse
cgaatagcaggctccaaccctgacc

SEQ ID NO: 8 - DNA, 21 bp, mouse
ccatgaatgccagcagctact

SEQ ID NO: 9 - DNA, 20 bp, mouse
cactgacacgcacacggact

SEQ ID NO: 10 - DNA, 19 bp, mouse
tgccgcaatgacggagccc

SEQ ID NO: 11 - DNA, 20 bp, mouse
ggagccatggattgcacatt

SEQ ID NO: 12 - DNA, 20 bp, mouse
cctgtctcacccccagcata

SEQ ID NO: 13 - DNA, 31 bp, mouse
cagctcatcaacaaccaagacagtgacttcc

SEQ ID NO: 14 - DNA, 20 bp, mouse
cctgaaccgcttctgggatt

SEQ ID NO: 15 - DNA, 20 bp, mouse
gctcttcctggacctggtca

SEQ ID NO: 16 - DNA, 23 bp, mouse
aaagcgtctgcacccagcgcagg

SEQ ID NO: 17 - DNA, 20 bp, mouse
gctctgctcattgccatcag

SEQ ID NO: 18 - DNA, 23 bp, mouse
tgttgcagcctctctactttgga

SEQ ID NO: 19 - DNA, 21 bp, mouse
tctgcagaccggcccaacgtg

SEQ ID NO: 20 - DNA, 20 bp, mouse
ggcatcattgggcactcctt

SEQ ID NO: 21 - DNA, 20 bp, mouse
gctgcaagcacagcctctct

SEQ ID NO: 22 - DNA, 26 bp, mouse
ccatctgcatcgccacaggcaacctc

SEQ ID NO: 23 - DNA, 20 bp, mouse
agagggaaatcgtgcgtgac

SEQ ID NO: 24 - DNA, 21 bp, mouse
caatagtgatgacctggccgt

SEQ ID NO: 25 - DNA, 23 bp, mouse
cactgccgcatcctcttcctccc

SEQ ID NO: 26 - DNA, 21 bp, mouse
gccgcgaggcactccagtgct

SEQ ID NO: 27 - DNA, 23 bp, mouse
cccagcaggatgcgcatggcgat

SEQ ID NO: 28 - DNA, 20 bp, artificial sequence
  primer consisting of nucelotides 1306-1325 of the ABCA1 promoter
ccacgtgctttctgctgagt

SEQ ID NO: 29 - DNA, 20 bp, artificial sequence
  primer consisting of nucelotides 1426-1445 of the ABCA1 promoter
tgccgcgactagttcctttt

SEQ ID NO: 30 - DNA, 24 bp, Homo sapiens
aaactggctatcattggagacgcg

(19) United States
(12) Patent Application Publication
(10) Pub. No.: US 2006/0009520 A1
(43) Pub. Date: Jan. 12, 2006

(54) Retinoid-based methods for altering macrophage cholesterol

(76) Inventors: Alan R. Tall, Cresskill, NJ (US); Florent LaLanne, Villefranche sur Saone (FR); Philippe Costet, Saint Orens de Gameville (FR)

Correspondence Address: COOPER & DUNHAM, LLP, 1185 AVENUE OF THE AMERICAS, NEW YORK, NY, 10036, US

(21) Appl. No.: 11/128,918

(22) Filed: May 12, 2005

Related U.S. Application Data
(60) Provisional application No. 60/570,576, filed on May 12, 2004.

(51) Int. Cl. A61K 31/203; A61K 31/192; A61K 31/07

(52) U.S. Cl. 514/559; 514/569; 514/725

(57) ABSTRACT

This invention provides methods for increasing cholesterol efflux from a macrophage and decreasing the amount of cholesterol in a macrophage, preferably using a retinoid. This invention also provides methods for increasing the survival time of a cholesterol-loaded macrophage and decreasing the likelihood that a cholesterol-loaded macrophage will contribute to the progression of atherosclerosis, preferably using a retinoid. Finally, this invention provides related therapeutic methods and articles of manufacture.

The invention disclosed herein was made with government support under Grant No. HL 22682 from the National Institutes of Health of the U.S. Department of Health and Human Services. Accordingly, the U.S. Government has certain rights in this invention.

Throughout this application, various publications are referenced by author and date. Full citations for these publications may be found listed alphabetically at the end of the specification immediately preceding the claims. The disclosures of these publications in their entireties are hereby incorporated by reference in order to more fully describe the state of the art.

### BACKGROUND OF THE INVENTION

The levels of high-density lipoprotein (HDL) in plasma are inversely related to the incidence of atherosclerotic cardiovascular disease, in part because of the ability of HDL and its apolipoproteins to mediate the efflux of cholesterol from macrophage foam cells (2). The molecular basis of apolipoprotein-mediated cholesterol efflux was recently elucidated by the discovery that Tangier disease, characterized by low HDL levels in plasma, macrophage foam cell accumulation, and increased atherosclerosis, is caused by mutations in the ATP-binding cassette transporter 1 (ABCA1). ABCA1 mediates efflux of phospholipids and cholesterol from cells to lipid-poor apolipoproteins, such as apoA-I and apoE, to form nascent HDLs (32, 36, 47).

ABCA1 is upregulated in cholesterol-loaded cells, as a result of increased transcription mediated by the oxysterol-activated nuclear receptors liver X receptor (LXR)/retinoid X receptor (RXR) acting on a direct repeat nuclear receptor binding site spaced by 4 nucleotides (DR4) in the proximal promoter of the ABCA1 gene (6, 31, 33). Treatment of animals with LXR activators reduces atherosclerosis, and bone marrow transplantation experiments indicate a specific antiatherogenic function of LXRs and ABCA1 in macrophages (16, 38). LXRs target a battery of genes mediating cholesterol efflux, transport and excretion and have emerged as major drug targets (5). However, LXRs also act at the promoter of sterol regulatory element-binding protein-1c (SREBP-1c), a master transcriptional regulator of genes of fatty acid and triglyceride synthesis, resulting in fatty liver and hypertriglyceridemia (3, 12, 30).

Vitamin A and its derivatives, the retinoids, exert many biological activities at different stages of development. They are crucial for the normal development of the embryo and are later essential for cell proliferation, differentiation, and apoptosis (7, 17). Two classes of nuclear receptors mediate these biological effects: RXRs and retinoic acid receptors (RARs). Each of these classes consists of three isoforms (α, β, and γ) (11, 24, 25, 29, 49). RXR is activated by 9-cis-retinoic acid (9-cRA), whereas RAR is activated by all-trans-retinoic acid (ATRA) and 9-cRA (1). In vivo, dimeric RXR/RAR typically binds to promoter elements consisting of direct repeats spaced by five nucleotides (DR5) (14). 13-cRA and ATRA are in clinical use, and retinoids are under active investigation for several different conditions.

### SUMMARY OF THE INVENTION

This invention provides a method for increasing cholesterol efflux from a macrophage comprising contacting the macrophage with an agent that increases the expression of a gene encoding a protein involved in the efflux, transport and/or absorption of cholesterol in the macrophage.

This invention further provides a method for decreasing the amount of cholesterol in a macrophage comprising contacting the macrophage with an agent that increases the expression of a gene encoding a protein involved in the efflux, transport and/or absorption of cholesterol in the macrophage.

This invention further provides a method for increasing cholesterol efflux from a macrophage comprising contacting the macrophage with a retinoid.

This invention further provides a method for decreasing the amount of cholesterol in a macrophage comprising contacting the macrophage with a retinoid.

This invention further comprises a method for increasing the likelihood that a cholesterol-loaded macrophage will survive comprising contacting the macrophage with an agent that increases the expression of a gene encoding a protein involved in the efflux, transport and/or absorption of cholesterol in the macrophage.

This invention further provides a method for decreasing the likelihood that a cholesterol-loaded macrophage will contribute to the progression of atherosclerosis in a subject comprising contacting the macrophage with an agent that increases the expression of a gene encoding a protein involved in the efflux, transport and/or absorption of cholesterol in the macrophage.

This invention further provides a method for increasing the likelihood that a cholesterol-loaded macrophage will survive comprising contacting the macrophage with a retinoid.

This invention further provides a method for decreasing the likelihood that a cholesterol-loaded macrophage will contribute to the progression of atherosclerosis in a subject comprising contacting the macrophage with a retinoid.

This invention further provides a method for treating a subject afflicted with atherosclerosis comprising administering to the subject a therapeutically effective amount of an agent that increases the expression of a gene encoding a protein involved in the efflux, transport and/or absorption of cholesterol in the subject's macrophages.

This invention further provides a method for treating a subject afflicted with atherosclerosis comprising administering to the subject a therapeutically effective amount of a retinoid.

This invention further comprises an article of manufacture comprising (a) a packaging material having therein an agent, wherein the agent increases the expression of a gene encoding a protein involved in the efflux, transport and/or absorption of cholesterol in a macrophage, and (b) a label indicating that the agent is intended for use in treating a subject afflicted with atherosclerosis.

Finally, this invention provides an article of manufacture comprising (a) a packaging material having therein a retinoid, and (b) a label indicating that the agent is intended for use in treating a subject afflicted with atherosclerosis.

### BRIEF DESCRIPTION OF THE FIGURES

FIGS. 1A-1D: Retinoids Induce ABCA1 in Macrophages

(A) Retinoids stimulate ABCA1-mediated cholesterol efflux from mouse peritoneal macrophages. The ability of macrophages to efflux cholesterol to apoA-I responds to ATRA treatment in a dose-dependent fashion. The results are expressed as mean±the standard error of the mean (SEM: n=4). *, P<0.05; **, P<0.01 (compared to control). (B) ATRA and TO-901317 (LXR agonist) induce a comparable increase in cholesterol efflux to apoA-I. The results are expressed as mean±the SEM (n=4). (C) Retinoids increase ABCA1 protein accumulation in macrophages. ABCA1 protein levels were analyzed by Western blot in mouse peritoneal macrophages and human monocyte-derived macrophages after ATRA treatment (10 μM for human macrophages) for 24 h in DMEM containing 10% lipoprotein-deficient serum. The fold induction is shown standardized against β-actin. (D) The synthetic RAR pan-agonist, TTNPB, also increases ABCA1 protein accumulation in macrophages.

FIGS. 2A and 2B: Induction of Macrophages Genes by ATRA

(A) Mouse peritoneal macrophages in DMEM containing 10% LPDS were treated for 24 h with various concentrations of ATRA (0.1 to 5 μM) or vehicle (DMSO). (B) Human monocyte-derived macrophages were treated for 24 h with various concentrations of ATRA (0.5 to 5 μM) or vehicle (DMSO). The expression of ABCA1, ABCG1, SREBP-1c, apoE, and LXRα mRNA were measured by quantitative real-time PCR assays (TaqMan) and standardized against β-actin mRNA levels. *, P<0.05; **, P<0.01; ***, P<0.001 (compared to control).

FIGS. 3A and 3B: Retinoids do not Induce Lipogenic SREBP-1c Target Genes In Vivo

(A) RAR regulation of ABCA1 and SREBP-1c expression in mouse peritoneal macrophages. Macrophages were exposed to TTNPB (1 μM) or DMSO (control) for 24 h in 10% LPDS. ABCA1 and SREBP-1c mRNA levels were determined by quantitative real-time reverse transcription-PCR and standardized against β-actin mRNA levels. The results are expressed as mean±the SEM (n=4 and 6). *, P<0.05; **, P<0.01 (compared to control). (B) Regulation of gene expression by TTNPB in the mouse liver. Mice were injected intraperitoneally with TTNPB (1 or 10 mg/kg) or vehicle (DMSO-polyethylene glycol 300). After 24 h, the mice were anesthetized, the livers were perfused, and the square lobes were removed for isolation of RNA. The expression of SREBP-1c, FAS, ABCA1, and Cyp26 mRNA (positive control for the effect of TTNPB) were measured by quantitative real-time PCR assays (TaqMan) and standardized against β-actin mRNA levels. The results are expressed as mean±the SEM (n=5).

FIGS. 4A-4F: Human ABCA1 Promoter is Activated by RXR/RAR

(A) In HEK293 cells, hABCA1 promoter is activated by RAR-γ. HEK293 cells were transfected with hABCA1 promoter (pb −928 to pb +101) and/or pCMX-hRXRα, pCMX-hRARα, pCMX-hRARβ, and PCMX-I1RARγ1 and then exposed to DMSO (control) or 0.1 μM TTNPB in DMEM-lipoprotein-deficient serum-10% penicillin-streptomycin for 36 h before analysis. The luciferase activity was determined as described previously (6). The values are means±the SEM of three to six independent experiments. *, P<0.05 (Mann-Whitney test). (B) Activation of human ABCA1 promoter by RARγ does not need cotransfection of RXRα. (C) RAR activates hABCA1 promoter through its LXRE DR4 element. HEK 293 cells were transfected with hABCA1 wild-type promoter, a deleted version (bp −100 to bp +101), or the full-length promoter containing mutations in the DR4 element previously described as an LXRE (6). Cells were cotransfected with pCMX-RXRα and pCMX RARγ1 and exposed to 0.1 μM TTNPB for 36 h before luciferase analysis. The values are mean±the SD of three independent experiments performed in duplicates. (D) RXRα/RARγ1 heterodimer binds hABCA1 DR4 element in EMSAs. In vitro-translated RXRα and RARγ were incubated with ³²P-labeled hABCA1 DR4 element. The arrow indicates the resulting complex. Lane 1, wheat germ extract; lane 2, RXRα/RARγ complex on the DR4; lane 3, competition with unlabeled hABCA1 DR4; lanes 4 and 5, asterisks represent a shift of the complex in the presence of RARγ (lane 4) or RXRα (lane 5) polyclonal antibody; lane 6, control anti-RORα antibody; lane 7, competition with mutated unlabeled hABCA1 DR4 (described in FIG. 4C, independent experiment). (E) Structure of the mSREBP-1c promoter and position of the two DR4s (LXRE a and b). (F) RXRα/RARγ heterodimer does not interact with the DR4 sequences of the mouse SREBP-1c promoter. (Left panel) RXRα, RARγ, and LXRβ were separately produced by using an in vitro transcription-translation wheat germ extract systems and used in EMSAs with a ³²P-labeled mouse SREBP-1c DR4b element as a probe. Lane 1, RXRα/LXRβ binding; lane 2, absence of binding of RXRα/RARγ. (Right panel) EMSA analysis as described in panel D but with a ³²P-labeled human ABCA1 DR4 element as a probe. Lane 1, binding of RXRα/RARγ on DR4; lane 2, competition with unlabeled probe; lane 3, competition assay using unlabeled mouse SREBP-1c DR4b element as competitor.

FIGS. 5A-5D:

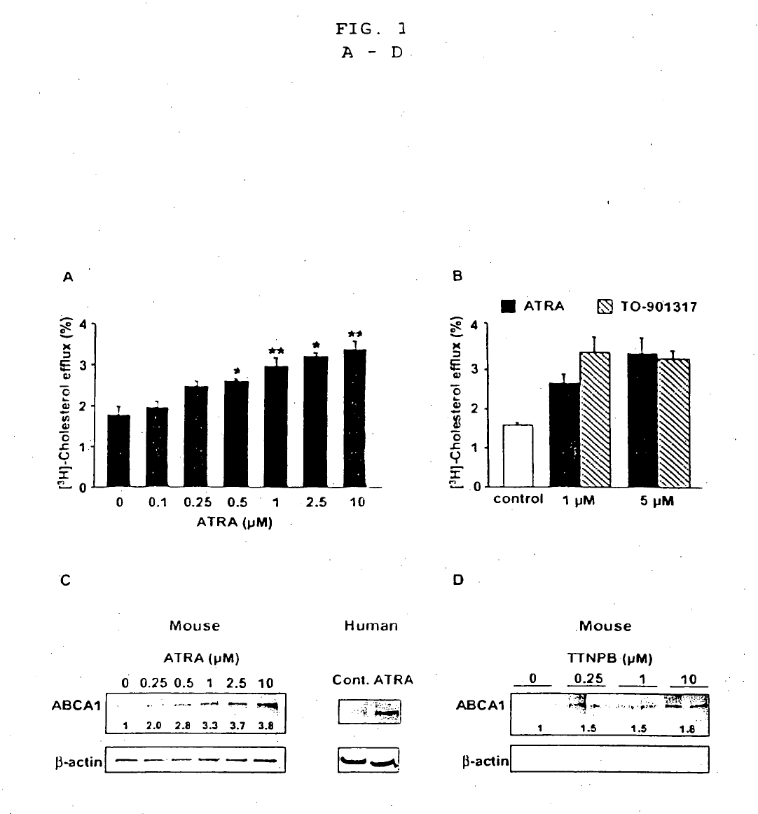

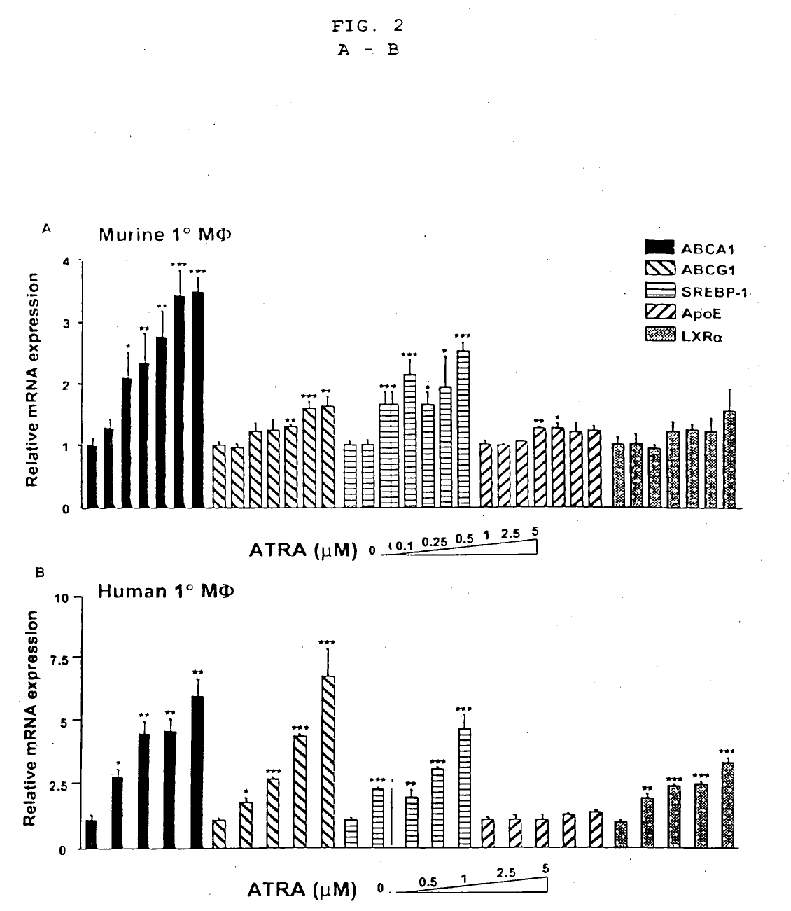

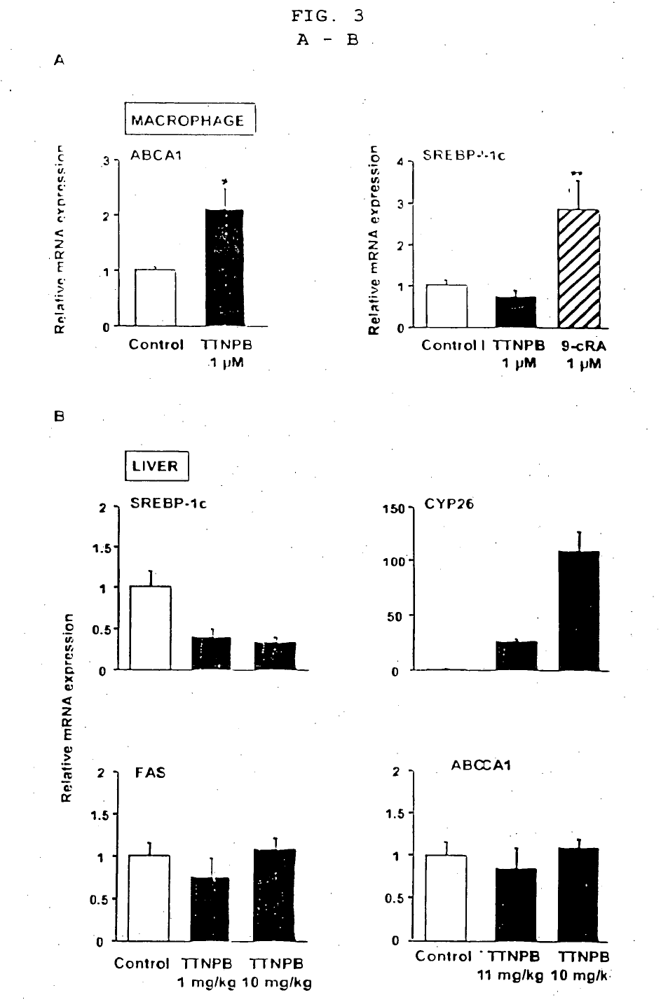

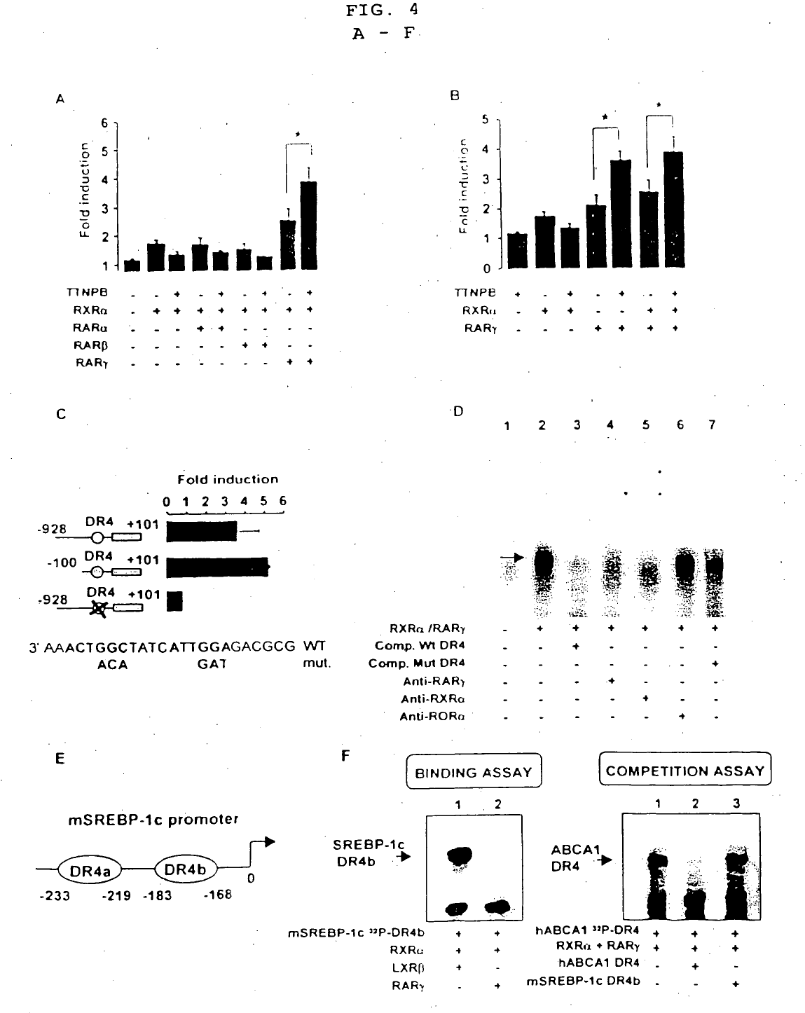

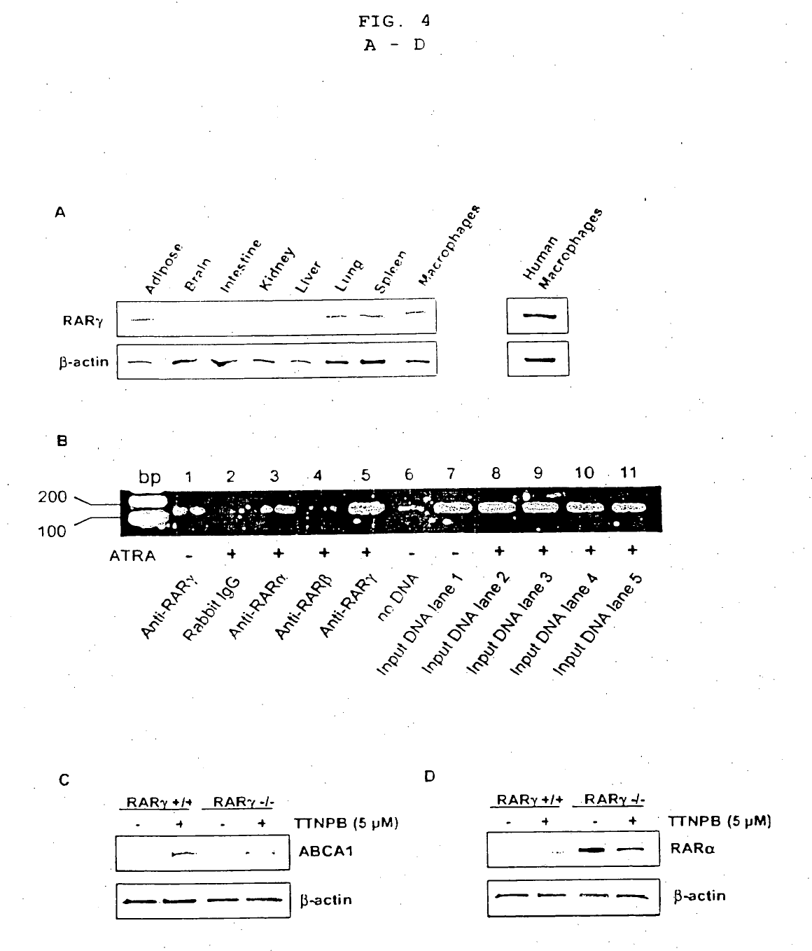

(A) RARγ tissue distribution in C57BL/6J mouse. Western blot analyses were performed after SDS-polyacrylamide gel electrophoresis separation of 100 μg of the nuclear proteins extracted from each tissue. (B) In vivo association of RARγ/RXR dimer with the DR4 region in the ABCA1 promoter as determined by ChIP analysis. Mouse peritoneal macrophages were treated or not treated (lane 1) with 1 μM ATRA for 24 h and subjected to ChIP assays. Lanes 1 and 5, rabbit anti-RARγ polyclonal antibody used for immunoprecipitation; lane 2, normal rabbit immunoglubulin G used for immunoprecipitation negative control; lane 3, rabbit anti-RARα polyclonal antibody used for immunoprecipitation; lane 4, rabbit anti-RARβ polyclonal antibody used for immunoprecipitation; lane 6, no DNA; lanes 7 to 12, input DNA used for PCR. (C) ABCA1 protein accumulates in TTNPB-treated RARγ^{-/-} mouse peritoneal macrophages. Thioglycolate-elicited peritoneal macrophages from RARγ^{-/-} and RARγ^{+/+} mice were treated with 5 μM TTNPB for 24 h in DMEM-10% lipoprotein-deficient serum-1% penicillin-streptomycin. The ABCA1 protein levels were then analyzed by Western blot analysis as described for FIG. 1C. (D) Upregulation of RARα in RARγ^{-/-} mouse peritoneal macrophages. Nuclear protein extracts isolated from RARγ^{-/-} and RARγ^{+/+} macrophages were separated by SDS-polyacrylamide gel electrophoresis, and the RARα protein level was determined by Western blot analysis.

### DETAILED DESCRIPTION OF THE INVENTION

### DEFINITIONS

“ABCA1” is used herein to mean “ATP-binding cassette transporter A1”, and is also referred to in the art as “ABC1”.

“Administering” may be effected or performed using any of the methods known to one skilled in the art. The methods comprise, for example, intralesional, intramuscular, subcutaneous, intravenous, intraperitoneal, liposome-mediated, transmucosal, intestinal, topical, nasal, oral, anal, ocular or otic means of delivery.

“Atherosclerosis” shall include, without limitation, atherosclerotic vascular disease, advanced atherosclerotic lesions, atherosclerosis-associated acute thrombosis, or other clinical events associated with the aforementioned complications.

As used herein, “cholesterol” includes, without limitation, esterified cholesterol (e.g., cholesteryl esters), and non-esterified cholesterol (e.g., free-cholesterol).

As used herein, “cholesterol efflux” shall mean the movement of cholesterol from a cell to the cell's exterior, and/or any biochemical step constituting part of such movement. In one embodiment, cholesterol is moved from a cell to a cholesterol acceptor which then transports the cholesterol out of the cell.

As used herein, a “cholesterol-loaded” cell shall mean a cell having a level of cholesterol higher than normal for that cell type. For example, if a human macrophage has a cholesterol level of X, and a human macrophage in question has a cholesterol level of 2X, the human macrophage in question is considered “cholesterol-loaded.” A higher than normal cholesterol level can be any level higher than normal including, for example, 1%, 2%, 5%, 10%, 20%, 50%, and 100% higher than normal. In one embodiment, free cholesterol-loaded cells are formed in culture by human intervention. This is accomplished, for example, by contacting the cells in culture with a cholesterol-containing particle, such as an acetylated low density lipoprotein, under conditions where ACAT is inhibited. If ACAT is not inhibited, then the cells become loaded primarily with cholesteryl esters instead of free cholesterol.

As used herein, “pharmaceutically acceptable carrier” means a carrier that is compatible with the other ingredients of a formulation and is not deleterious to the recipient thereof. Such carriers include, for example, 0.01-0.1 M and preferably 0.05 M phosphate buffer or 0.8% saline. Additionally, pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Retinoid” shall include, without limitation, vitamin A and various synthetic or naturally occurring analogs thereof.

“Subject” shall mean any organism including, without limitation, a mammal such as a mouse, a rat, a dog, a guinea pig, a ferret, a rabbit and a primate. In the preferred embodiment, the subject is a human being.

“Therapeutically effective amount” means an amount sufficient to treat a subject afflicted with a disorder or a complication associated with a disorder. The therapeutically effective amount will vary with the subject being treated, the condition to be treated, the agent delivered and the route of delivery. A person of ordinary skill in the art can perform routine titration experiments to determine such an amount. Depending upon the agent delivered, the therapeutically effective amount of agent can be delivered continuously, such as by continuous pump, or at periodic intervals (for example, on one or more separate occasions). Desired time intervals of multiple amounts of a particular agent can be determined without undue experimentation by one skilled in the art.

“Treating” means either slowing, stopping or reversing the progression of a disorder. As used herein, “treating” also means ameliorating symptoms associated with a disorder.

### EMBODIMENTS OF THE INVENTION

This invention provides a method for increasing cholesterol efflux from a macrophage comprising contacting the cell with an agent that increases the expression of a gene encoding a protein involved in the efflux, transport and/or absorption of cholesterol in the macrophage. The protein can be, but is not limited to, ABCA1, CYP27A1, CETP, Apoprotein E, and/or LXR. In the preferred embodiment, the agent is a retinoid. In another embodiment, the agent is TTNPB or trans-retinoic acid.

This invention further provides a method for decreasing the amount of cholesterol in a macrophage comprising contacting the macrophage with an agent that increases the expression of a gene encoding a protein involved in the efflux, transport and/or absorption of cholesterol in the macrophage.

This invention further provides a method for increasing cholesterol efflux from a macrophage comprising contacting the macrophage with a retinoid. In one embodiment, the retinoid is TTNPB or trans-retinoic acid.

This invention further provides a method for decreasing the amount of cholesterol in a macrophage comprising contacting the macrophage with a retinoid. In one embodiment, the retinoid is TTNPB or trans-retinoic acid.

This invention further comprises a method for increasing the likelihood that a cholesterol-loaded macrophage will survive comprising contacting the macrophage with an agent that increases the expression of a gene encoding a protein involved in the efflux, transport and/or absorption of cholesterol in the macrophage. The protein can be, but is not limited to, ABCA1, CYP27A1, CETP, Apoprotein E, and/or LXR. In the preferred embodiment, the agent is a retinoid. In one embodiment, the agent is TTNPB or trans-retinoic acid. In the above instant methods, the agent (e.g. retinoid) contacted with the macrophage is in an amount effective to bring about the stated result. Such amounts can be determined by one of ordinary skill in the art without undue experimentation.

This invention further provides a method for decreasing the likelihood that a cholesterol-loaded macrophage will contribute to the progression of atherosclerosis in a subject comprising contacting the macrophage with an agent that increases the expression of a gene encoding a protein involved in the efflux, transport and/or absorption of cholesterol in the macrophage. The protein can be, but is not limited to, ABCA1, CYP27A1, CETP, Apoprotein E, and/or LXR. In the preferred embodiment, the agent is a retinoid. In another embodiment, the agent is TTNPB or trans-retinoic acid.

This invention further provides a method for increasing the likelihood that a cholesterol-loaded macrophage will survive comprising contacting the macrophage with a retinoid. In one embodiment, the retinoid is TTNPB or trans-retinoic acid.

This invention further provides a method for decreasing the likelihood that a cholesterol-loaded macrophage will contribute to the progression of atherosclerosis in a subject comprising contacting the macrophage with a retinoid. In one embodiment, the retinoid is TTNPB or trans-retinoic acid.

This invention further provides a method for treating a subject afflicted with atherosclerosis comprising administering to the subject a therapeutically effective amount of an agent that increases the expression of a gene encoding a protein involved in the efflux, transport and/or absorption of cholesterol in the subject's macrophages. The protein can be, but is not limited to, ABCA1, CYP27A1, CETP, Apoprotein E, and/or LXR. In the preferred embodiment, the agent is a retinoid. In another embodiment, the agent is TTNPB or trans-retinoic acid. In the preferred embodiment, the subject is a human. Also in a preferred embodiment, the agent is admixed with a pharmaceutically acceptable carrier.

This invention further provides a method for treating a subject afflicted with atherosclerosis comprising administering to the subject a therapeutically effective amount of a retinoid. In one embodiment, the retinoid is TTNPB or trans-retinoic acid. In a preferred embodiment, the subject is a human. Also in a preferred embodiment, the retinoid is admixed with a pharmaceutically acceptable carrier.

This invention further comprises an article of manufacture comprising (a) a packaging material having therein an agent, wherein the agent increases the expression of a gene encoding a protein involved in the efflux, transport and/or absorption of cholesterol in a macrophage, and (b) a label indicating that the agent is intended for use in treating a subject afflicted with atherosclerosis. In one embodiment, the protein is ABCA1, CYP27A1, CETP, Apoprotein E, and/or LXR. In the preferred embodiment, the agent is a retinoid. In another embodiment, the agent is TTNPB or trans-retinoic acid. Also, in the preferred embodiment, the subject is a human.

Finally, this invention provides an article of manufacture comprising (a) a packaging material having therein a retinoid, and (b) a label indicating that the agent is intended for use in treating a subject afflicted with atherosclerosis. In one embodiment, the retinoid is TTNPB or trans-retinoic acid. In a preferred embodiment, the subject is a human.

This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### SYNOPSIS

In the present study, a possible role of retinoids in the regulation of macrophage cholesterol efflux and ABCA1 gene expression was examined. It was found that RAR ligands, ATRA and TTNPB (4-[E-2-5,6.7.8-tetrahydro-5,5,8,8-tetramethyl-2-naphthalenyl-l-propenyl]benzoic acid), upregulate the ABCA1 gene, unexpectedly acting at the noncanonical DR4 element of the ABCA1 promoter. These studies suggest a broader role of retinoids acting through a specific RAR isoform (RARγ) in the regulation of macrophage functions, including cholesterol efflux and transport.

### MATERIALS AND METHODS

Reagents

ATRA (Sigma. St. Louis. Mo.), TO-OOI317 (Sigma). TTNPB (Bio-Mol Research Laboratories, Inc., Plymouth Meeting, Pa.), and 9-cRA (BioMol) were dissolved in dimethyl sulfoxide (DMSO; Sigma).

Animals

Male C57BL/6J mice (Jackson Laboratory, Bar Harbor, Me.) were housed in a temperature- and light-controlled facility. RARγ^{-/-} mice and their control littermates RARγ^{+/+} were maintained on a C57BL/6J genetic background and genotyped by Southern blot. Mice were aged matched for each experiment. All animal procedures were approved by the Institutional Animal Cure and Research Advisory Committee at Columbia University.

Cell Cultures and Transfection Experiments

Human HEK293 cells were purchased from the American Type Culture Collection (Manassas, Va.) and maintained in Dulbecco modified Eagle medium (DMEM) with 10% fetal bovine serum, 100 U of penicillin/ml, and 100 mg of streptomycin/ml. Primary peritoneal macrophages were isolated from C57BL/6J male mice 6 to 8 weeks old intra-peritoneally injected with 1 ml of 30% thioglycolate. After 72 h, macrophages were collected by washing the peritoneal cavity with phosphate-buffered saline (PBS) and cultured in DMEM medium supplemented with 10% fetal bovine scrum, 100 U of penicillin/ml, and 100 mg of streptomycin/ml for 48 h before the experiment. Human monocyte-derived macrophages were prepared and cultured as described previously (10).

Transfection experiments were performed in 24-well plates with Lipofectamine Plus reagent according to the manufacturer's instructions (Invitrogen). Cells were transfected with 12.5 ng of phRLTK [Renilla; Promega, Madison, Wis.)/well, 0.15 μg of reporter DNA (containing hABCA1 proximal promoter)/well, and 0.15 μg of each receptor (pCMX-hRXRα, pCMX-hRARα, pCMX-hRARβ, and pCMX-hRARγ)/well and pcDNA3.1 (to a final total of 0.45 μg/well) if necessary. The transfected cells were cultured in DMEM with 10% lipoprotein-deficient serum, 100 U of penicillin/ml, and 100 mg of streptomycin/ml in the presence of 0.1 μM TTNPB or its vehicle for 36 h. Luciferase activity was then measured by using the Dual Luciferase assay system (Promega) and normalized with Renilla.

Cholesterol Efflux Assays

Macrophages were cholesterol loaded and radiolabeled overnight in DMEM 0.2% bovine scrum albumin (BSA; Sigma) containing 50 μg of acetylated low-density lipoprotein and 1 μCi of [³H]cholesterol (51.2 Ci/mmol; NEN/Life Science Products, Boston, Mass.)/ml in the presence or absence of ATRA or TO-901317. Cells were washed with PBS, equilibrated for 30 min in DMEM-0.2% BSA, and then incubated for 4 h in the efflux media containing DMEM-0.2% BSA and 10 μg of purified apoA-1/ml in the presence or absence of the different ligands. The media was then collected, and cells were lysed with a 0.1% NaOH-0.1% sodium dodecyl sulfate (SDS) solution. After determination of the radioactivity recovered in the medium and cell lysate by liquid scintillation counting, cholesterol efflux was calculated as the percentage of the radioactivity recovered in the media over the total radioactivity (cells plus media) after subtraction of the nonspecific apoA-1-free media. Cholesterol efflux assays were performed in triplicates or quadruplicates.

Western Blot Analysis

Protein extracts from macrophages were prepared by lysing the cells in modified radioimmunoprecipitation assay buffer (50 mM Tris, 150 mM NaCl, 1 mM EDTA. 1% Triton X-100) containing a protease inhibitor cocktail (Complete EDTA Free; Roche). Protein content in the extracts was determined by using the DC protein assay (Bio-Rad, Hercules, Calif.). Equal amounts of protein (40 to 50 μg) were separated by electrophoresis with 4 to 15% acrylamide gradient gels (Bio-Rad) and then transferred to nitrocellulose membrane (Trans-Blot Transfer Medium; Bio-Rad). Membranes were probed with anti-ABCA1 antibody (Novus Biologicals, Littleton, Colo.) and anti-β-actin antibody (Sigma) according to the manufacturer's recommendations. Immunoblots were developed by using a chemiluminescent detection system (Super Signal West Pico chemiluminescent substrate; Pierce, Rockford, Ill.).

Nuclear protein extracts from tissues and macrophages were isolated according to the following method. Tissue samples and cells were first homogenized on ice and then lysed in lysis buffer (10 mM Tris [pH 7.5], 3 mM MgCl₂, 10 mM NaCl, 0.5% NP-40) containing protease inhibitor cocktail. The nucleus was then pelleted by centrifugation for 10 min at 6,000× g, immediately resuspended in a nucleus suspension buffer (250 mM Tris [pH 8.0], 60 mM KCl, I mM dithiothrietol), and then rotated for 1 h. After centrifugation for 5 min at 6000× g, the solubilized nuclear proteins were separated by electrophoresis with a 4 to 15% gradient gel (Bio-Rad), transferred to nitrocellulose membrane (Trans-Blot transfer medium; Bio-Rad), and probed with anti-RARγ and anti-RARα antibodies (Santa Cruz Biotechnology, Santa Cruz, Calif.) and their respective horseradish peroxidase-conjugated secondary antibodies according to the manufacturer's recommendations. The intensities of the bands were quantified by using ImageQuant and normalized to (β-actin.

EMSA

RARs and LXRα and RXRα proteins were in vitro translated by using the TNT-coupled wheat germ extract systems (Promega). Double-strand oligonucleotides corresponding to the wild-type ABCA1 DR4 element 5′-ACTGGGCTTTGACCGATAGTAACCTCTGCGCTCG-3′ and the mutated sequence 5′-ACTGGGCTTTGTGTGATAGTACTATCTGCGCTCG-3′ were used, and electrophoresis mobility shift assays (EMSAs) were done with ³²P-labeled probes as described previously (6). For competition experiments, a 30-fold molar excess of cold unlabeled competitor DNA relative to labeled DNA was used. In antibody experiments, the mixture was first incubated for 10 min at room temperature with 0.4 to 1 μg of anti-RARγ or anti-RXRα rabbit polyclonal antibody (Santa Cruz Biotechnology). The oligonucleotides 5′-ACTGCAGTGACCGCCAGTAACCCCAGC-3′ and 5′-ACTGGGACGCCCGCTAGTAACCCCGGC-3′ were, respectively, used in EMSA analysis of DR4 elements a and b of the murine SREBP-1c promoter.

Plasma and Hepatic Lipid Analysis

Mice were fasted for 3 h before blood collection. Plasma was separated by centrifugation and kept at −80° C. until lipid analysis. Liver tissue samples (50 to 75 mg) were homogenized in PBS. Lipids were then extracted with chloroform-methanol (2/1 [vol/vol]) and redissolved in isopropanol. Triglyceride and cholesterol were measured in plasma and in the liver lipid extracts by using commercial kits (Wako Chemicals, Neusse Germany).

RNA Analysis

Total RNA was isolated from mouse peritoneal macrophages or ˜50 mg of mouse liver tissue by using the RNeasy Mini kit (Qiagen, Valencia, Calif.) or RNA-Bee reagent (Tel-Test, Inc., Friendswood, Tex.), respectively, according to the manufacturer's protocol. Real-time quantitative PCR assays were performed by using the Mx4000 Quantitative PCR System (Stratagene, La Jolla, Calif.). Briefly, 5 μg of total RNA was treated with RNase-free DNase I (Ambion, Austin, Tex.), and first-strand cDNA was synthesized with oligo(dT)₁₂₋₁₈ by using a Superscript II RNase H⁻ reverse transcriptase reagent kit (Invitrogen) according to the manufacturer's protocol. For quantification of mouse ABCA1, ABCG1, SREBP-1c, LXRα, ApoE, and fatty acid synthase (FAS) mRNA levels, each amplification mixture contained 62.5 ng of cDNA, appropriate concentrations of forward and reverse primers and of dual-labeled fluorogenic probe (Biosearch Technologies, Novato, Calif.), and 12.5 μl of TaqMan Universal PCR master mix (Applied Biosystems, Foster City, Calif.). PCR thermocycling parameters were 50° C. for 2 min, 95° C. for 10 min, and 45 cycles of 95° C. for 15 s and 60° C. for 1 min. All samples were analyzed for β-actin expression in the same run. Quantitative expression values were extrapolated from standard curves for the gene of interest with 10-fold dilutions of cDNA (in triplicate). Each sample was normalized to β-actin, triplicates were averaged, and relative mRNA levels were determined. The following mouse primers and probes were used: mouse ABCA1 (mABCA1) forward (F) (5′-GGTTTGGAGATGGTTATACAATAGTTGT-3′), mABCA1 reverse (R) (5′-CCCGGAAACGCAAGTCC-3′), and mABCA1 TaqMan probe (5′-FAM-CGAATAGCAGGCTCCAACCCTGACC-BHQ-3′); mABCG1 F (5′-CCATGAATGCCAGCAGCTACT-3′), mABCG1 R (5′-CACTGACACGCACACGGACT-3′), and mABCG1 TaqMan probe (5′-FAM-TGCCGCAATGACGGAGCCC-BHQ-3′); mSREBP-1c F (5′-GGAGCCATGGATTGCACATT-3′), mSREBP-1c R (5′-CCTGTCTCACCCCCAGCATA-3′), and mSREBP-1c TaqMan probe (5′-FAM-CAGCTCATCAACAACCAAGACAGTGACTTCC-BHQ-3′); mApoE F (5′-CCTGAACCGCTTCTGGGATT-3′), mApoE R (5′-GCTCTTCCTGGACCTGGTCA-3′), and mApoE TaqMan probe (5′-FAM-AAAGCGTCTGCACCCAGCGCAGG-BHQ-3′); mLXRα F (5′-GCTCTGCTCATTGCCATCAG-3′), mLXRα R (5′-TGTTGCAGCCTCTCTACTTTGGA-3′), and mLXRα TaqMan probe (5′-FAM-TCTGCAGACCGGCCCAACGTG-BHQ-3′); mFAS F (5′-GGCATCATTGGGCACTCCTT-3′), mFAS R (5′-GCTGCAAGCACAGCCTCTCT-3′), and mFAS TaqMan probe (5′-FAM-CCATCTGCATAGCCACAGGCAACCTC-BHQ-3′); and mβ-actin F (5′-AGAGGGAAATCGTGCGTGAC-3′), mβ-actin R (5′-CAATAGTGATGACCTGGCCGT-3′), and mβ-actin TaqMan probe (5′-JOE-CACTGCCGCATCCTCTTCCTCCC-BHQ-3′).

For quantification of mouse Cyp26 mRNA levels, each amplification mixture (25 μ) contained 62.5 ng of cDNA, 100 nM concentrations of reverse and forward primers, and 2.5 μl of 10× SYBR Green PCR master mix (Perkin-Elmer Life Sciences, Boston, Mass.). Quantitative expression values were extrapolated from standard curves for Cyp26 expression with 10-fold dilutions of cDNA (in triplicate). Each sample was normalized to β-actin that was deduced from TaqMan assays, triplicate results were averaged, and relative Cyp26 mRNA levels were determined. The primers mCyp26 F (5′-GCCGCGAGGCACTCCAGTGCT-3′) and mCyp26 R (5′-CCCAGCAGGATGCGCATGGCGAT-3′) were used. RNA measurements from human monocyte-derived macrophages were performed as described previously (10, 44).

Chromatin Immunoprecipitation (ChIP) Assays

Mouse peritoneal macrophages were treated or not with 1 μM ATRA for 24 h and then incubated with 1% formaldehyde in cell culture media for 20 min. Thereafter, cells were washed twice with ice-cold PBS and lysed in buffer containing 1% SDS, 10 mM EDTA, 50 mM Tris-HCl (pH 8.1), and protease inhibitor cocktail (Roche). Samples were sonicated three times with 10-s pulses at 4° C. After centrifugation the samples were diluted 1:10 in buffer containing 0.01% SDS, 1.1% Triton X-100, 1.2 mM EDTA, 16.7 mM Tris-HCl (pH 8.1), and 167 mM NaCl and precleared with 80 μl of salmon sperm DNA-protein A-agarose for 2 h at 4° C. Samples were then centrifuged to pellet the agarose beads, and immunoprecipitation was performed on the supernatant by using anti-RARα, anti-RARβ, anti-RARγ polyclonal antibodies or an equal amount of normal rabbit immunoglobulin G (Santa Cruz Biotechnology) overnight at 4° C. The antibody-protein-DNA complexes were then precipitated by 60 μl of salmon sperm DNA-protein A-agarose for 1 h at 4° C. The precipitates were washed sequentially in low-salt immune complex buffer (0.1% SDS, 1% Triton X-100, 2 mM EDTA, 20 mM Tris-HCl [pH 8.1], 150 mM NaCl), hight-salt immune complex buffer (0.1% SDS. 1% Triton X-100, 2 mM EDTA, 20 mM Tris-HCl [pH 8.1], 500 mM NaCl), LiCl immune complex buffer (0.25 mM LiCl, 1% NP-40, 1% deoxycholate, 1 mM EDTA, 10 mM Tris-HCl [pH 8.11) prior to two final washes in Tris-EDTA buffer. The protein-DNA complexes were eluted by using a 1% SDS-0.1 M NaHCO₃ solution. Cross-linked DNA was reversed by incubation at 65° C. for 6 h in presence of 5 M NaCl, and proteins were digested at 45° C. for 1 h with proteinase K. Immunoprecipitated DNA fragments were purified by using a QIAquick PCR purification kit (Qiagen). Samples were analyzed by PCR with the primers 5′-CCACGTGCTTTCTGCTGAGT-3′ and 5′-TGCCGCGACTAGTTCCTTTT-3′ (nucleotides 1306 to 1325 and 1426 to 1445 of the ABCA1 promoter; GenBank accession number AF275948).

### RESULTS

RAR Activators Induce ABCA1 and Increase Macrophage Cholesterol Efflux

To determine whether ABCA1-mediated cholesterol efflux is increased by RAR activators, primary cultures of mouse peritoneal macrophages were treated with the naturally occurring RAR ligand ATRA and the efflux of cholesterol to apoA-1 was measured. ATRA increased ABCA1-dependent cholesterol efflux to apoA-1 in a dose-dependent fashion (FIG. 1A). The maximum dose of 10 μM increased the efflux by ˜2-fold. At doses of 1 and 5 μM ATRA, the increase in cholesterol efflux was comparable to that occurring in macrophages treated with the LXR activator TO-901317 (FIG. 1B). ABCA1 protein levels were dose dependently increased after ATRA treatment, paralleling effects on cholesterol efflux (FIG. 1C). ABCA1 protein was also increased in human primary monocytes/macrophages treated with ATRA (FIG. 1C). To determine whether the increase in ABCA1 protein and cholesterol efflux might be caused by induction of ABCA1 gene expression, ABCA1 mRNA was measured by quantitative real-time PCR. This revealed an increase in the mRNA that was parallel to the dose response of protein and cholesterol efflux (FIG. 2A), a finding consistent with increased gene expression as the underlying mechanism.

Induction of LXR Target Genes by ATRA in Mouse and Human Macrophages

The response to ATRA of a panel of genes involved in cholesterol efflux and lipid metabolism in both mouse and human primary macrophages cultures was assessed (FIG. 2). These genes were chosen because they are targets of LXR/RXR in macrophages (18, 19, 30, 41). In mouse primary macrophages the response to ATRA was fairly specific for ABCA1 (FIG. 2A), with a clear dose relationship and responses at doses as low as 0.25 μM ATRA. In contrast, there was no significant induction of apoE or LXRα, and a weaker and somewhat inconsistent induction of ABCG1 and SREBP-1c (FIG. 2A). Human primary monocytes/macrophages showed a more general induction of LXR target genes by ATRA. There was a strong induction of both ABCA1 and ABCG1 and weaker but significant increases in LXRα and SREBP-1c. However, compared to these other genes, the fold induction was more pronounced for ABCA1 at lower doses of ATRA (0.5 and 1 μM), a difference that was observed in repeated experiments. ApoE, another macrophage LXR/RXR target (19, 23), was not induced by ATRA either in human or mouse primary macrophages even at the higher dose of ATRA.

TTNPB Induces Macrophage ABCA1 but not SREBP-1c

ATRA is a ligand for RARs but not RXRs (15). However, there may be a small amount of spontaneous conversion of ATRA to other retinoids, such as 9-cRA, that are ligands for RXRs (15), raising the possibility that some of the alterations in gene expression in response to ATRA might reflect activation of RXRs. Thus, mouse macrophages were treated with TTNPB, a synthetic RAR pan-agonist that does not activate RXRs (24). This resulted in accumulation of ABCA1 protein, which is similar to the effects of ATRA (FIG. 1D). There was also an induction of macrophage ABCA1 mRNA by TTNPB, but no effect on SREBP-1c mRNA (FIG. 3A). Thus, TTNPB causes a specific induction of mouse macrophage ABCA1, strongly suggesting a response mediated via RARs.

SREBP-1c, a known LXR target gene (30), induces expression of genes involved in fatty acid and triglyceride synthesis, leading to fatty liver after administration of LXR activators (12). To see whether RAR activators might have the potential to induce ABCA1 and cholesterol efflux in macrophages without inducing fatty liver, mice were injected intraperitoneally with TTNPB and the expression of genes involved in lipogenesis in the liver was measured. The known RAR target gene, Cyp26 (22), was markedly induced by TTNPB in liver (FIG. 3B). However, there was no response of the FAS gene, and hepatic SREBP-1c mRNA was actually repressed after TTNPB injection. Consistent with these observations, treatment of mice with intraperitoneal TTNPB did not result in an increase in hepatic triglyceride content and was associated with a significant reduction in triglyceride levels in plasma at the higher doses (Table 1).

TABLE 1
Comparison of plasma and hepatic lipid parameters
of mice treated with TTNPB or its vehicle^{a}

| | | | Hepatic lipid | |
|---|---|---|---|---|
| | Plasma lipid (mg/dl) | | (μg/mg of tissue) | |
| Treatment | Triglycerides | Cholesterol | Triglycerides | Cholesterol |
| Control | 39.8 ± 3.4 | 58.6 ± 6.2 | 12.05 ± 2.01 | 1.44 ± 0.12 |
| TTNPB | | | | |
| (mg/kg) | | | | |
| 1 | 35.1 ± 4.0 | 64.1 ± 8.0 | 11.57 ± 4.05 | 1.43 ± 0.07 |
| 10 | 33.1 ± 3.1 | 58.0 ± 4.9 | 9.25 ± 1.98 | 1.31 ± 0.20 |

^{a}Retinoids do not induce either hypertriglyceridemia or fatty liver in vivo. Mice were injected intraperitoneally with TTNPB or vehicle (DMSO-polyethylene glycol 300 [control]). Twenty-four hours later, cholesterol and triglyceride contents were measured in plasma and liver samples.

In part, these results may reflect the fact that TTNPB is a retinoid-like molecule that also activates FXR at relatively high concentrations (48), possibly resulting in decreased triglyceride synthesis in liver. Hepatic ABCA1 was not induced in animals treated with TTNPB (FIG. 3B). This could reflect alternative ABCA1 promoter usage (4) or possibly the lack of specific RAR isoforms in the liver (see below). These results suggest that RAR activators have the potential to induce macrophage ABCA1 without causing fatty liver.

The effects of TTNPB in human macrophages were also examined. TTNPB was much less effective than ATRA at inducing ABCA1 or other LXR target genes (not shown). However, when the response of Cyp26 was measured, the response to TTNPB was also much less pronounced in human macrophages (the mRNA increase was ˜6-fold versus 30-fold in murine macrophages), suggesting the metabolism of TTNPB in human macrophages.

RARγ/RXR Activates the ABCA1 Promoter via a DR4 Element

To further assess the possibility of a direct activation of the ABCAI promoter by RARs, human ABCA1 promoter-reporter constructs were used in transfection studies and treated the cells with TTNPB. HEK293 cells were transfected with the human ABCA1 promoter (bp −928 to bp +101) linked to luciferase, as well as each of the three different isoforms of RAR (α, β, and γ) and RXRα (FIG. 4A). Although RARα and RARβ failed to activate the ABCA1 promoter, either in presence or the absence of TTNPB, RXRα/RARγ increased luciferase activity by 2.8-fold in the basal state and by 4.0-fold in the presence of TTNPB. Transactivation of the ABCA1 promoter by RARγ was not dependent on cotransfection with RXR (FIG. 4B), indicating that RARγ activates the ABCA1 promoter, either by acting as a homodimer or as a heterodimer with endogenous RXR.

To locate the RAR response element, mutational analysis of the human ABCAI promoter was performed (FIG. 4C). HEK293 cells were transfected with various constructs containing the full-length human ABCA1 promoter (bp −928 to bp +101) or a deleted version (bp −100 to bp +101), together with RARγ and RXRα, in the presence of TTNPB. A construct containing the full-length promoter containing point mutations in the DR4 element that are known to abolish the interaction with RXR/LXR was also included (6). Deleting the region from bp −928 to bp −101 had no effect on the hABCA1 promoter response to retinoids. Surprisingly, the mutation in the DR4 element abolished the RARγ-mediated activation of the hABCA1 promoter. This suggests that the DR4 element mediates the response of the ABCA1 promoter to RAR7 and retinoids.

To further assess the possibility of a direct interaction between RARγ and the human ABCA1 promoter, a gel shift assay by using oligonucleotides consisting of the DR4 element was carried out. RARγ/RXRα formed a specific complex on the human ABCA1 promoter (FIG. 4D, lane 2) that was specifically competed by an unlabeled oligonucleotide containing the DR4 element (lane 3) but not by a mutant DR4 element (lane 7). Antibodies raised against RXRα or RARγ abolished the complex and gave rise to supershifted complexes (lanes 4 and 5, asterisks), indicating that the complex consists of RARγ/RXRα. An antibody raised against ROR, an irrelevant nuclear receptor, had no effect (lane 6). It was also determined whether RARα and RARβ could bind the ABCA1 DR4 element. Each of the three RAR isoforms formed a specific complex on the ABCA1 DR4 element but only in the presence of RXRα (data not shown), confirming that complexes are heterodimers of RAR/RXR. These results show direct binding of RAR/RXR to the ABCA1 promoter, involving the same DR4 promoter element that binds LXR/RXR. In contrast to the transactivation assay in transfected, 293 cells (FIG. 4A), the binding was not specific for a particular RAR isoform. This could indicate that specificity in the transactivation assay depends on RAR isoform-specific sets of coactivators or corepressors present in HEK 293 cells.

It was also sought to determine whether there might be a comparable binding of RAR/RXR to the promoters of other LXR target genes. The SREBP-1c promoter contains two LXR/RXR binding sites (FIG. 4E). However, neither element bound to RARγ/RXRα or competed with the RARγ/RXRα complex formed on the ABCA1 DR4 element (FIG. 4F shows data for the DR4b element) or bound any other RAR isoform (not shown). This finding suggests that some of the genes more weakly induced by ATRA (FIG. 2) may be indirect targets.

ChIP of RARγ in Mouse Macrophages

Further experiments were carried out to verify a direct effect of RARs on the mouse macrophage ABCA1 DR4 promoter element. First, to evaluate the expression of RARγ protein in different mouse tissues, Western blots were performed on nuclear extracts. This revealed a high level of expression of RARγ in spleen, adipose and lung, with much lower levels in liver, intestine and kidney (FIG. 5A). RARγ protein was also well expressed in primary macrophage cultures from mice and humans. RARα was found to be highly expressed in adipose tissue, lung, and spleen, whereas RARβ was widely expressed in different tissues (data not shown), similar to the distribution of their cognate mRNAs (49). Whereas RARβ protein was not detected in murine macrophages (not shown), RARα protein was detected at low levels in wild-type macrophages (FIG. 5D).

ChIP assays of the ABCA1 promoter revealed a specific binding of RARγ to the DR4 element (FIG. 5B, lane 5).

There was a much weaker signal for RARα (lane 3) and no signal for RARβ (lane 4). Similar specific binding of RARγ was observed for three different macrophage preparations. Interestingly, the signal for of RARγ was much stronger when cells were treated with ATRA (FIG. 5B, lane 1 versus lane 5). In other experiments, it was observed that incubation of macrophages with ATRA weakly induced RARγ protein (<2-fold) (data not shown), suggesting a predominant effect of ATRA on RARγ binding to the DR4 element rather than an induction of RARγ itself.

Response of ABCA1 in RARγ-Deficient Macrophages

To further evaluate a possible specific role of RARγ in the upregulation of ABCA1, macrophages from RARγ^{-/-} mice were examined (21). Recovery of thioglycolate-elicited macrophages was considerably lower in RARγ^{-/-} mice than in controls (ca. ⅓ the number of cells), but cellular morphology appeared similar to that seen with controls, perhaps indicating a role of RARγ in the migration of macrophages into tissues. To evaluate the upregulation of ABCA1, macrophages were treated with 5 μM TTNPB. This experiment showed that ABCA1 was still induced in RARγ^{-/-} macrophages, a finding similar to the results in macrophages from RARγ^{+/+} controls (FIG. 5C). Similar results were obtained in two separate experiments performed on macrophages pooled from four mice per group. Interestingly, nuclear RARα was substantially increased (˜20-fold) in RARγ-deficient macrophages (FIG. 5D) and decreased in response to TTNPB treatment (˜2-fold). These findings suggest autoregulation of RAR expression, both between and within RAR isoforms. Most likely the upregulation of RARα compensates for the deficiency of RARγ, leading to induction of ABCA1 expression by TTNPB. Even though RARα did not increase ABCA1 promoter activity in HEK293 cells, it is possible that the cell-specific context in macrophages allows a response to increased RARα, albeit weaker than the RARγ response, a finding consistent with the ChIP assay results.

### DISCUSSION

This study reports that RAR activators induce robust ABCA1 gene expression in mouse and human primary macrophages, and that this is mediated at least in part by a direct effect of RARγ/RXR on the ABCA1 promoter. Even though the effect is mediated via the LXR-binding site in the ABCA1 promoter, other LXR target genes, including SREBP-1c, were modestly induced by ATRA, possibly by indirect mechanisms. RARγ is highly expressed in macrophages but not highly expressed in liver (FIG. 5A). These findings suggest a role of RARγ in the regulation of macrophage cholesterol efflux via ABCA1.

Surprisingly, the effects of RARγ/RXR on the promoter of ABCA1 were found to be mediated via a noncanonical DR4 element, previously implicated in the activity of LXR/RXR (6). This initially raised the possibility that effects of ATRA on ABCA1 gene expression could reflect conversion to other retinoids such as 9-cRA with subsequent activation of RXR in LXR/RXR complexes. However, multiple lines of evidence accrued to indicate a direct action of RARγ/RXR on the DR4 element, including transactivation and gel shift assays and, most compellingly, direct demonstration of binding in ChIP analysis of the ABCA1 promoter in macrophages. Moreover, macrophage ABCA1 was induced by TTNPB, a synthetic agonist that is specific for RAR and not RXR. A number of other LXR target genes were evaluated and showed weak or no induction in mouse macrophages but stronger induction in human macrophages. Thus, it is possible that in part the effect of ATRA in human macrophages reflects induction of LXRα, as suggested in a report that appeared while the present study was under review (43), or conversion to other retinoids that act on RXR. However, even in human macrophages induction of A13CA1 was more prominent than that of other LXR target genes at lower doses, a finding consistent with a direct effect of RAR on the human ABCA1 promoter, as shown in the transactivation and gel shift assays (FIG. 4A). Noncanonical binding of RARγ/RXR to a DR4 element has been described (39, 40), although the functional implications of such binding have not been previously shown.

Transactivation assays in HEK293 cells and ChIP analysis in macrophages indicated a selective effect of RARγ/RXR complexes on the ABCA1 promoter, a finding consistent with the high expression of RARγ in macrophages. However, there was a weak signal above background for RARα in the ChIP analysis of the ABCA1 promoter, and induction of ABCA1 by TTNPB in macrophages from RARγ^{-/-} mice indicated that the effect on ABCA1 was not completely specific for the RARγ isoforms (FIG. 5C). This likely reflected a 20-fold upregulation of RARα in RARγ-deficient macrophages. These findings indicate partial compensation between the different RAR isoforms in relation to macrophage functions consistent with the evidence of such compensation in embryos from mice with knockouts of the various RAR isoforms (20, 21, 28). This functional redundancy between RARγ and RARα has also been shown in RARγ-null F9 cells, where basal expression of RARα did not induce RARγ responsive genes, but responsiveness of RARγ target genes was observed when RARα was overexpressed (37). The ability of ABCA1 to respond to RARα in macrophages but not HEK293 cells could potentially reflect the presence of different sets of coregulators in the different cell types and may explain the modest effects of RARs on the ABCA1 promoter in HEK293 cells. Although these studies have focused on the role of RARγ activators in ABCA1 gene expression, RARγ may have an essential role in macrophage differentiation and function, as suggested by the low recovery of macrophages in RARγ-deficient mice. Interestingly, RARγ is also highly expressed in adipocytes and forced expression in preadipocytes blocks the program of adipocyte differentiation (46). Further studies are indicated to define the more general roles of RARγ in macrophage differentiation and functions.

These findings suggest convergent signaling of retinoids and oxysterols on the macrophage ABCA1 promoter. A convergence of RAR and LXR signaling pathways might be related to events occurring in the embryo, involving first RAR and later LXR. Mice lacking the RARγ gene, together with one or both copies of RARβ display severe interdigital webbing associated with a low number of apoptotic cells and an increase of cell proliferation in the interdigital necrotic zone (8). Mouse embryos deficient in ABCA1 also exhibit delayed clearance of interdigital webbing accompanied by the accumulation of apoptotic corpses (13). Interestingly, ABCA1 appears to be the human homolog of Caenorhabditis elegans ced-7, which functions in engulfment of cell corpses during apoptosis (45). Chimini and coworkers have suggested that ABCA1 promotes engulfment of apoptotic cells (13). Therefore, it is possible that ABCA1 levels in macrophages might be controlled by retinoids at the initiation of the tissue remodeling process and later by cellular sterol content, reflecting ongoing phagocytosis of cholesterol-rich corpses.

Current clinical use of retinoids is related to their properties as cellular differentiating agents. 13-cRA is used to treat severe cystic acne (9, 34). ATRA induces remissions in ca. 80% of patients with acute promyelocytic leukemia. Retinoids are promising chemopreventive agents, and clinical studies have also demonstrated their effectiveness in reversing premalignant lesions, such as leukoplakia, and in preventing second primary tumors of the head and neck and also liver and breast cancer (35). ATRA is also in clinical trials for the treatment of emphysema (26).

A major concern in the use of retinoids has been the induction of hypertriglyceridemia, often accompanied by reduced HDL levels, as well as elevations of transaminase in plasma, probably resulting from the development of fatty liver. The molecular mechanisms of these side effects are not well understood and might be due to the low specificity of the retinoids used. For some retinoids, they may be related to activation of RXR, induction of SREBP-1c, and genes of fatty acid synthesis. Other mechanisms of dyslipidemia may be related to induction of apoCIII. Indeed, 13-cRA (isotretinoin) increases hepatic apoCIII expression at a transcriptional level, providing an explanation for hypertriglyceridemia (42). apoCIII delays the catabolism of triglyceride-rich particles and increases atherosclerosis (27). Vu-dac et al. showed that this regulation is mediated by RXR and not by RAR (42). These studies indicate a modest induction of SREBP-1c by ATRA and suggest that this might be a mechanism underlying the fatty liver and hypertriglyceridemia associated with clinical use of this agent.

### REFERENCES

1. Allenby, G., et al., “Retinoic acid receptors and retinoid X receptors: Interaction with endogenous retinoic acids.” Proc. Natl. Acad. Sci. USA 90:30-34, (1993).
2. Attie, A. D., et al., “Pivotal role of ABCA1 in reverse cholesterol transport influencing HDL levels and susceptibility to atherosclerosis.” J. Lipid Res. 42:1717-1726, (2001).
3. Cao, G., et al., “Phospholipid transfer protein is regulated by liver X receptors in vivo.” J. Biol. Chem. 277:39561-39565, (2002).
4. Cavelier, L. B., et al., “Regulation and activity of the human ABCA1 gene in transgenic mice.” J. Biol. Chem. 276: 18046-18051, (2001).
5. Chawla, A., et al., “Nuclear receptors and lipid physiology: opening the X files.” Science 294: 1866-1870, (2001).
6. Costet, P., et al., Sterol-dependent transactivation of the ABC1 promoter by the liver X receptor/retinoid X receptor.” J. Biol. Chem. 275: 28240-28245, (2000).
7. De Luca, L. M., “Retinoids and their receptors in differentiation, embryogenesis, and neoplasia.” FASEB J. 5:2924-2933, (1991).
8. Dupe, V., et al., “Essential roles of retinoic acid signaling in interdigital apoptosis and control of BMP-7 expression in mouse autopods.” Dev. Biol. 208: 30-43, (1999).
9. Farrell, L. N., et al., “The treatment of severe cystic acne with 13-cis-retinoic acid: evaluation of sebum production and the clinical response in a multiple-dose trial.” J. Am. Acad. Dermatol. 3: 602-611, (1980).
10. Fu, X., et al., “27-Hydroxycholesterol is an endogenous ligand for liver X receptor in cholesterol-loaded cells.” J. Biol. Chem. 276: 38378-38387, (2001).
11. Giguere, V., et al., “Identification of a receptor for the morphogen retinoic acid.” Nature 330: 624-629, (1987).
12. Grefhorst, A., et al., “Stimulation of lipogenesis by pharmacological activation of the liver X receptor leads to production of large, triglyceride-rich very low density lipoprotein particles.” J. Biol. Chem. 277: 34182-34190, (2002).
13. Hamon, Y., et al., “ABC1 promotes engulfment of apoptic cells and transbilayer redistribution of phosphatidylserine.” Nat. Cell. Biol. 2: 399-406, (2000).
14. Heery, D. M., et al., “Homo- and heterodimes of the retinoid X receptor (RXR) activated transcription in yeast.” Nucleic Acids Res. 22: 726-731, (1994).
15. Heyman, R. A., et al., “9-cis-Retinoic acid is a high-affinity ligand for the retinoid X receptor.” Cell 68: 397-406, (1992).
16. Joseph, S. B., et al., “Synthetic LXR ligand inhibits the development of atherosclerosis in mice.” Proc. Natl. Acad. Sci. USA 99: 7604-7609, (2002).
17. Konta, T., et al., “Selective roles of retinoic acid receptor and retinoid X receptor in the suppression of apoptosis by all-trans-retinoic acid.” J. Biol. Chem. 276: 12697-12701, (2001).
18. Laffitte, B. A., et al., “Autoregulation of the human liver X receptor alpha promoter.” Mol. Cell. Biol. 22:7558-7568, (2001).
19. Laffitte, B. A., et al., “LXRs control lipid-inducible expression of the apolipoprotein E gene in macrophages and adipocytes.” Proc. Natl. Acad. Sci. USA 98: 507-512, (2001).
20. Li, E., et al., “Normal development and growth of mice carrying a targeted disruption of the α1 retinoic acid receptor gene.” Proc. Natl. Acad. Sci. USA 90: 1590-1594, (1993).
21. Lohnes, D., et al., “Function of retinoic acid receptor gamma in the mouse.” Cell 73: 643-658, (1993).
22. Loudig, 0., et al., “Cytochrome P450RAI (CYP26) promoter: a distinct composite retinoic acid response element underlies the complex regulation of retinoic acid metabolism.” Mol. Endocrinol. 12: 1483-1497, (2000).
23. Mak, P. A., et al., “Regulated expression of the apolipoprotein E/C—I/C—IV/C—II gene cluster in murine and human macrophages: a critical role for nuclear liver X receptors alpha and beta.” J. Biol. Chem. 277: 31900-31908, (2002).
24. Mangelsdorf, D., et al., “Nuclear receptor that identifies a novel retinoic acid response pathway.” Nature 345: 224-229, (1990).
25. Mangelsdorf, D., et al., “The RXR heterodimes and orphan receptors.” Cell 83: 841-850, (1995).
26. Mao, J. T., et al., “A pilot study of all-trans-retinoic acid for the treatment of human emphysema.” Am. J. Respir. Crit. Care Med. 165: 718-723, (2002).
27. Masucci-Magoulas, L., et al., “A mouse model with features of familial combined hylerlipidemia.” Science 275: 391-394, (1997).
28. Mendelsohn, C., et al., “Retinoic acid receptor β2 (RARβ2) null mutant mice appear normal.” Dev. Biol. 166: 246-258, (1994).
29. Petkovich, M., et al., “A human retinoic acid receptor which belongs to the family of nuclear receptors.” Nature 330: 444-450, (1987).
30. Repa, J. J., et al., Regulation of mouse sterol regulatory element-binding protein-1c gene (SREBP-1c) by oxysterol receptors, LXRα and LXRβ.” Genes Dev. 14: 2819-2830, (2000).
31. Repa, J. J., et al., “Regulation of absorption and ABC1-mediated efflux of cholesterol by RXR heterodimers.” Science 289: 1524-1529, (2000).
32. Rothblat, G. H., et al., “Cell cholesterol efflux: integration of old and new observations provides new insights.” J. Lipid Res. 40: 781-796, (1999).
33. Sparrow, C. P., et al., “A potent synthetic LXR agonist is more effective than cholesterol loading at inducing ABCCA1 mRNA and stimulating cholesterol efflux.” J. Biol. Chem. 277: 10021-10027, (2002).
34. Spear, K. L. and Muller, S. A., “Treatment of cystic acne with 13-cis-retinoic acid.” Mayo Clin. Proc. 58: 509-514, (1983).
35. Sun, S. Y. and Lotan, R., “Retinoids and their receptors in cancer development and chemoprevention.” Crit. Rev. Oncol. Hematol. 41: 41-55, (2002).
36. Tall, A. R., et al., “Regulation and mechanism of macrophages cholesterol efflux.” J. Clin. Investig. 110: 899-904, (2002).
37. Taneja, R., et al., “Reexpression of retinoic acid receptor (RAR) γ or overexpression of RARα or RARβ in RAR γ-null F9 cells reveals a partial functional redundancy between the three RAR types.” Proc. Natl. Acad. Sci. USA 92: 7854-7858, (1995).
38. Tangirala, R. K., et al., “Identification of macrophage liver X receptors as inhibitors of atherosclerosis.” Proc. Natl. Acad. Sci. USA 99: 11896-11901, (2002).
39. Umesono, K., et al., “Direct repeats as selective response elements for the thyroid hormone, retinoic acid, and vitamin D₃ receptors.” Cell 65: 1255-1266, (1991).
40. Vasios, G., et al., “The late retinoic acid induction of laminin B1 gene transcription involves RAR binding to the responsive element.” EMBO J. 10: 1149-1158, (1991).
41. Venkateswaran, A., et al., “Human white/murine ABC8 mRNA levels are highly induced in lipid-loaded macrophages: a transcriptional role for specific oxysterols.” J. Biol. Chem. 275: 14700-14707, (2000).
42. Vu-Dac, N., et al., “Retinoids increases human apoCIII expression at the transcriptional level via the retinoid X receptor.” J. Clin. Investig. 102: 625-632, (1998).
43. Wagsater, D., et al., “Induction of ATP-binding cassette A1 by all-trans retinoic acid: possible role of liver X receptor-alpha.” Int. J. Mol. Med. 4: 419-423, (2003).
44. Wright, S. D., et al., “Tumor-promoting phorbol esters stimulate C3b and Csb′ receptor-mediated phagocytosis in cultured human monocytes.” J. Exp. Med. 156: 1149-1164, (1982).
45. Wu, Y. C. and Horvitz, H. R., “The Caenorhabditis elegans cell corpse engulfment gene ced-7 encodes a protein similar to ABS transporters.” Cell 93: 951-960. (1998).
46. Xue, J. C., et al., “Distinct stages in adipogenesis revealed by retinoid inhibition of differentiation after induction of PPARγ.” Mol. Cell. Biol. 4: 1567-1575 (1998).
47. Yokoyama, S., “Apolipoprotein-mediated cellular cholesterol efflux.” Biochim. Biophys. Acta. 1392: 1-15, (1998).
48. Zavacki, A. M., et al., “Activation of the orphan receptor RIP14 by retinoids.” Proc. Natl. Acad. Sci. USA 94: 7909-7914, (1997).
49. Zelent, A., et al., “Cloning of murine alpha and beta retinoic acid receptors and a novel receptor gamma predominantly expressed in skin.” Nature 339: 714-717, (1989).

1. A method for increasing cholesterol efflux from a macrophage comprising contacting the macrophage with an agent that increases the expression of a gene encoding a protein involved in the efflux, transport and/or absorption of cholesterol in the macrophage.

2. A method for decreasing the amount of cholesterol in a macrophage comprising contacting the macrophage with an agent that increases the expression of a gene encoding a protein involved in the efflux, transport and/or absorption of cholesterol in the macrophage.

3-6. (canceled)

7. A method for decreasing the amount of cholesterol in a macrophage comprising contacting the macrophage with a retinoid.

8-34. (canceled)
